(19) United States
(12) Patent Application Publication
(10) Pub. No.: US 2006/0110422 A1
(43) Pub. Date: May 25, 2006

(54) Conversion of calcite powders into macro- and microporous calcium phosphate scaffolds for medical applications

(76) Inventors: Ahmet Cuneyt Tas, Central, SC (US); Sarit B. Bhaduri, Anderson, SC (US)

Correspondence Address: DORITY & MANNING, P.A., POST OFFICE BOX 1449, GREENVILLE, SC, 29602-1449, US

(21) Appl. No.: 10/993,225

(22) Filed: Nov. 19, 2004

(51) Int. Cl. A61K 33/42; A61F 13/00; A61K 38/39

(52) U.S. Cl. 424/422; 424/602

(57) ABSTRACT

Disclosed is a method for forming carbonated calcium hydroxyapatite. The disclosed method can be used for forming bone cements or optionally cast, cured scaffolds such as may be used in many medical applications, including as implants for bone damage caused by, for instance, trauma, disease, or surgical excision due to disease. The cured materials include an interconnected network including both microporosity and macroporosity. The disclosed materials can be very similar in both chemical and physical make-up to natural bone mineral. The invention is also directed to systems that can be used for conveniently carrying out the disclosed methods.

### BACKGROUND OF THE INVENTION

Synthetic, implantable bone-like materials are useful in many different medical applications. For example, bone-like scaffolding material can be implanted to fill large bone defects caused by trauma situations or excisement of cancerous or otherwise diseased bone. Ideally, such scaffolding would be formed to have a structure and composition compatible with that of natural bone. For instance, the ideal material should have a chemical and structural design so as to induce a response similar to that of fracture healing when placed in an osseous defect, including initial invasion by mesenchymal cells, fibroblasts and osteoblasts before new trabeculae of bone infiltrate into the porous structure of the implant from the walls of the defect. In particular, the chemical make-up of the implant should include calcium hydroxyapatite (Ca₁₀(PO₄)₆(OH)₂) and ideally, would include at least some carbonated hydroxyapatite (Ca₉(HPO₄, CO₃)(PO₄)₅(OH,CO₃)), so as to more closely resemble the chemical make-up of natural bone mineral. In addition, the porous structure of the implant would ideally include an interconnected porosity similar to that of natural bone.

Numerous techniques have been developed for the production of implantable apatitic calcium phosphate materials, which, unfortunately, typically involve a step of high-temperature (1050° C. to 1250° C.) sintering or calcination at the end of the manufacturing flowchart. Unfortunately, any carbonate ions that may be present in the calcium phosphate materials will volatilize when the processing temperature exceeds about 700° C. Also, calcium phosphates produced by many known methods exhibit high crystallinity and as such display poor bone bonding and remodeling ability in vivo, as human bones do not interact well with highly crystallized ceramics. In addition, even in those techniques wherein porous scaffolding materials have been formed, the porosity does not mimic that of natural bone, i.e., including both microporosity and macroporosity as is found in natural bone.

Other problems with existing processes include both the cost and the complexity of the methods. For example, most known processes require the use of calcium phosphate powders that must possess certain strict physical and chemical characteristics such as particle size, particle shape, surface reactivity, surface area, and chemical composition. These powders are manufactured on a small scale involving complicated, often tedious processing steps. As a result, these materials tend to be very expensive.

Many processes also require the use of materials that must be removed from the formed scaffolds in later processing steps. For example, some processes require the use of a sacrificial template to obtain at least some form of porosity. Other processes require the use of sacrificial porogens, such as sugar, salt, sodium bicarbonate, sodium acetate, gelatin, chitosan, and the like, which must be removed from the implants following formation. Moreover, these formation processes still fail to form porous networks including interconnected macro- and microporosity as is found in natural bone.

What is needed in the art are bone-like implantable materials that can more closely mimic natural bone material in chemical and structural design. In addition, what is needed in the art are simpler, more cost-effective methods for forming bone-like implantable materials.

### SUMMARY

In one embodiment, the present invention is directed to a method for forming carbonated hydroxyapatite. In general, the method includes mixing a calcium carbonate powder with an aqueous solution. In one particular embodiment, the calcium carbonate powder can be calcite. The aqueous solution can include sodium ions and phosphate ions and can have a pH between 3 and 6. The calcium carbonate powder and the aqueous solution can generally be combined in a ratio of between about 20 g powder per 30 ml solution and about 35 g powder per 30 ml solution. In one embodiment, the calcium carbonate powder can be combined with the aqueous solution in a ratio of about 1 g powder per ml solution. Upon combining these two components, they can be mixed for about 30 seconds to form carbon dioxide and a paste. More specifically, the paste can include a phase mixture of carbonated hydroxyapatite and calcium carbonate.

The paste formed according to the method can be used as formed as a bone cement or optionally, can be cast in a mold to form a macroscopic scaffold structure. In any case, the paste can cure either in vivo or ex vivo within about 5 minutes of application or casting. For instance, the paste can self-cure at ambient temperature within about 5 minutes. As such, the combination including the powder and the liquid components can generally be mixed for a relatively short period of time, for instance between about 30 seconds and about 90 seconds. In one embodiment, an additive can be added to the aqueous solution, for example an additive such as ethanol or denatured collagen, and the combination can be mixed for between about 30 seconds and about 240 seconds before casting the mixture in a mold or applying the mixture to a surface as a bone cement.

The carbon dioxide formed upon mixing the aqueous solution with the calcium carbonate powder can form an interconnected porous network throughout the paste. In particular, the porous network can include both microporosity and macroporosity.

Following cure, in one embodiment the cured scaffold can be soaked in water. For example, the scaffold can be soaked in water at ambient temperature for between about 10 hours and about 16 hours.

Additional processing of the scaffold can also be carried out. For instance, the scaffold can be soaked in a second solution comprising sodium ions and phosphate ions at physiological pH (i.e., about 7.4). During soaking, this solution can be heated to a temperature of between about 70° C. and about 100° C. For instance, the scaffold can be soaked in this solution for at least about 24 hours. In one embodiment, the scaffold can be soaked in this solution for between about 24 hours and about 48 hours. This second solution can also include additional materials, if desired. For example, the second solution can include a buffer or a biologically active agent. In one embodiment, the second solution can include a biologically active agent that can be loaded into the porous network of the scaffold structure as the scaffold soaks in the second solution.

Following initial formation, the scaffold can be packaged, if desired, as for storage, shipping, and the like. In one embodiment, the formed scaffolds can be used as is, or if desired, they can be subject to further formation, such as by cutting, prior to use, such as prior to implantation in a bone defect.

In one embodiment, the disclosed invention is directed to scaffolds that can be formed according to the disclosed processes. For example, the macroscopic scaffolds (i.e., defining a minimum cross-sectional dimension of at least one centimeter) can include a scaffold structure including carbonated, partially crystalline hydroxyapatite. The scaffold structure can define the interconnected porous network including both microporosity and macroporosity. For example, the microporosity can define pores of between about 1 μm and about 10 μm and the macroporosity can define pores of between about 300 μm and about 900 μm.

In one embodiment, the scaffold structure can be entirely formed of a two phase mixture of carbonated, partially crystalline hydroxyapatite and calcium carbonate. In another embodiment, all of the calcium carbonate can be converted to hydroxyapatite, and the scaffold structure can consist entirely of carbonated, partially crystalline hydroxyapatite.

The carbonated, partially crystalline hydroxyapatite of the scaffolds can, in one embodiment, include between about 1% and about 10% by weight carbonate ion. In one embodiment, the hydroxyapatite can include between about 2% and about 6% by weight carbonate ion.

In another embodiment, the disclosed invention is directed to a system capable of utilization in carrying out the disclosed methods. For instance, the system can include a first container for holding an amount of calcium carbonate powder and a second container for holding the aqueous solution for mixing with the powder. In particular, the containers can be sized to carry predetermined amounts of the two components so as to provide the two components in a ratio of between about 20 g powder per 30 ml solution and about 35 g powder per 30 ml solution.

Optionally, the disclosed systems can include additional components. For instance, the system can include a mixing element such as a spatula or the like. In one embodiment, the system can include another container of a volume so as to be capable of combining and mixing the two components in this container, i.e., a suitably sized mixing container. In one embodiment, the system can include a container for holding a soaking solution that is at physiological pH as herein described for soaking the scaffold and converting remaining calcium carbonate in the scaffold to hydroxyapatite. Optionally, the system can also include a mold for casting the paste and molding the scaffold.

### BRIEF DESCRIPTION OF THE FIGURES

A full and enabling disclosure of the present invention, including the best mode thereof, to one of ordinary skill in the art, is set forth more particularly in the remainder of the specification, including reference to the accompanying figures, in which:

FIGS. 1A-1E are electron microscope micrographs of increasing magnification of carbonated calcium hydroxyapatite scaffolds formed according to the presently disclosed processes;

FIG. 2 illustrates X-ray diffraction data of calcium hydroxyapatite scaffolds formed according to the presently disclosed processes;

FIG. 3 illustrates Fourier Transform Infrared (FTIR) Spectroscopy data of calcium hydroxyapatite scaffolds formed according to the presently disclosed processes;

FIG. 4 is a photograph of a carbonated hydroxyapatite scaffold formed according to the disclosed processes; and

FIG. 5 schematically illustrates a self-contained system according to one embodiment of the invention.

### DETAILED DESCRIPTION OF PREFERRED EMBODIMENTS

Reference will now be made in detail to various embodiments of the invention, one or more examples of which are illustrated in the accompanying Figures. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used with another embodiment to yield a still further embodiment. Thus, it is intended that the present invention cover such modifications and variations as come within the scope of the appended claims and their equivalents.

In one embodiment, the present invention is directed to methods for forming carbonated, partially crystalline hydroxyapatite such as may be utilized for forming bone-like scaffolds for medical applications. The present invention is also directed to systems that can be utilized for carrying out the disclosed methods, as well as to scaffolds that can be formed according to the disclosed methods. For purposes of the present disclosure, the term scaffold is herein defined to refer to macroscopically sized materials that can be shaped during and/or following formation so as to conform to a desired spatial orientation. For example, the scaffolds of the present invention can generally include exterior dimensions of at least about one centimeter in length. Hydroxyapatite scaffolds as herein defined are not to be confused with small, often microscopic, granules and particles including hydroxyapatite that have been formed in the past. Previously, such granules and particles have been found confined within a matrix so as to form an implantable multi-phasic macroscopic material, but they are not, in and of themselves, scaffolds as herein defined.

The processes of the present invention can provide simple, low temperature, and relatively inexpensive methods for the conversion of readily available calcium carbonate powders into implantable materials such as pre-formed scaffolds containing partially crystalline hydroxyapatite. Beneficially, the entire process can be carried out at low temperatures that can prevent the volatilization of carbonate ions formed during the process and as such, the process can form carbonated calcium hydroxyapatite materials closely resembling natural bone mineral in chemical make-up.

In addition to forming materials that closely resemble natural bone in chemical composition, the disclosed processes can also generate materials that closely resemble natural bone in physical structure. In particular, the disclosed materials can include a bone-like interconnected porous network that includes both micro-porosity and macro-porosity. The presence of such porosity can be extremely important, for instance so as to provide wicking characteristics and invite ingrowth of bone into the implanted scaffold. Porous structures exhibiting both macro- and microporosity can be particularly favorable when utilized in conjunction with natural cancellous bone, as they can closely mirror the structure of the host bone.

The unique combination of bone-like physical and chemical characteristics of the disclosed materials can facilitate bone growth in applications in which the materials are implanted in conjunction with existing natural bone, for example in bone repair applications. In addition, the disclosed materials can facilitate healing of bone defects due to trauma or surgical procedure. In particular, the disclosed products can support, foster, and facilitate bone growth when utilized in medical applications.

Calcium carbonate (CaCO₃) is the most common nonsiliceous mineral. While calcium carbonate naturally occurs in no less than three polymorphic forms including trigonal calcite, hexagonal vaterite, and orthorhombic aragonite, the calcite form of calcium carbonate is by far the most common and stable mineral of all of the calcium carbonate polymorphs. As such, in one preferred embodiment, calcite powder can be utilized as a starting material for the disclosed processes, but it should be understood that the present invention is not limited to the utilization of calcite powder, and in other embodiments, other calcium carbonate powders or mixtures of calcium carbonate powders can optionally be utilized including powders formed of any or any combination of aragonite, nacre, or vaterite.

According to the disclosed process, a calcium carbonate powder can be mixed with a liquid. The powdered component need have no particular particle size, shape, reactivity, or surface area. For example, in one embodiment, commercially available calcite powder, such as that available from Fisher Scientific, Inc. of Fairlawn, N.J., USA or Merck KGaA of Darmstadt, Germany, can be used directly as obtained.

The liquid component that can be combined with the calcite powder is an aqueous solution that contains phosphate ions. More particularly, the aqueous solution can include the phosphate ions, HPO₄²⁻ and/or H₂PO₄⁻.

In one embodiment, the liquid component can be formed by neutralization of a phosphoric acid solution with a strong base, such as a sodium hydroxide solution. As such, in addition to the phosphate ions, the liquid component can also include sodium ions, Na⁺. While utilization of sodium hydroxide as the neutralizing component may be preferred in some embodiments, it is not a requirement of the present invention. In particular, in many embodiments, sodium hydroxide can be preferred over other bases, such as potassium hydroxide, for example, since sodium will generally be more compatible with in vivo bone environments than will potassium. For example, in one embodiment, a concentrated (e.g., about 85 vol %) H₃PO₄ solution can be neutralized by a concentrated (e.g., about 50 vol %) NaOH solution by titration until the resultant solution has a pH value of between about 3 and about 6 to form the liquid component of the invention. In one embodiment, the liquid component can be formed so as to have a pH of between about 3.5 and about 5.3. In one particular embodiment, the liquid component can be formed so as to have a pH of about 5.

The liquid component as herein described can be a stable liquid capable of long-term storage. For instance, liquid preparations as described herein have been held in storage for over one year with no variation in pH or appearance noted. In particular, the liquid component can be stable against long-term storage at a wide range of temperatures: For example, the liquid component can be safely stored at temperatures over a range of between about 5° C. and about 50° C. over long periods of time with no deterioration of the chemical properties of the liquid.

According to the disclosed process, the powdered component and the liquid component can be combined in a ratio of between about 20 g powder per 30 ml solution and about 35 g powder per 30 ml solution. In one embodiment, the calcium carbonate powder can be combined with the aqueous solution liquid component in a ratio of about 1 g powder per milliliter solution. The combination can then be mixed at ambient temperature to form a foaming paste that can be cast to the form of a product scaffold or otherwise applied to a target location prior to final cure. Beneficially, the paste can cure at room temperature fairly rapidly. For example, in one embodiment, the paste can begin to set within about 90 seconds of mixing. Accordingly, the two components can be mixed for a short period of time, such as between about 30 seconds and about 90 seconds in some embodiments, and then applied to a target location as a paste or optionally cast or formed into the desired scaffold shape.

In another embodiment, the onset of the cure of the paste can be delayed somewhat through addition of an additive to the liquid component. For example, in one embodiment, a small amount, for example between about 2% and about 5% by volume of the liquid component can be an additive, such as ethanol or gelatin (i.e., denatured collagen) that can be added to the liquid component in order to delay the onset of the cure of the paste. For instance, in one embodiment, upon mixing calcite powder with a liquid component including an additive, the mixed paste can begin to set after about 240 seconds. Thus, according to this embodiment, a larger window of opportunity is available for forming the paste into the final shape. This particular embodiment of the invention may be preferred in those applications wherein the formed scaffold has a more complicated shape or in applications in which it may be beneficial to delay the onset of the cure, so as to ensure suitable time for proper application of the paste to the target. For instance, a slightly delayed cure may be beneficial in those embodiments wherein the paste is used in a surgical procedure to fill a bone defect or is applied as a bone cement in those applications wherein the paste can cure in vivo.

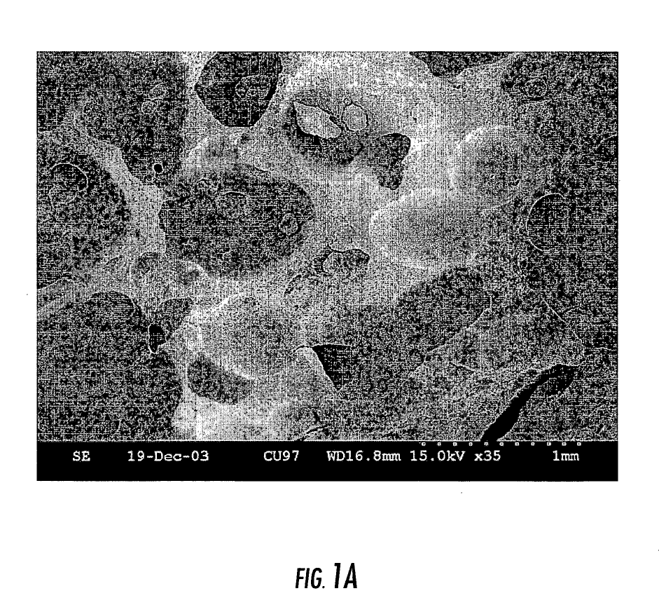

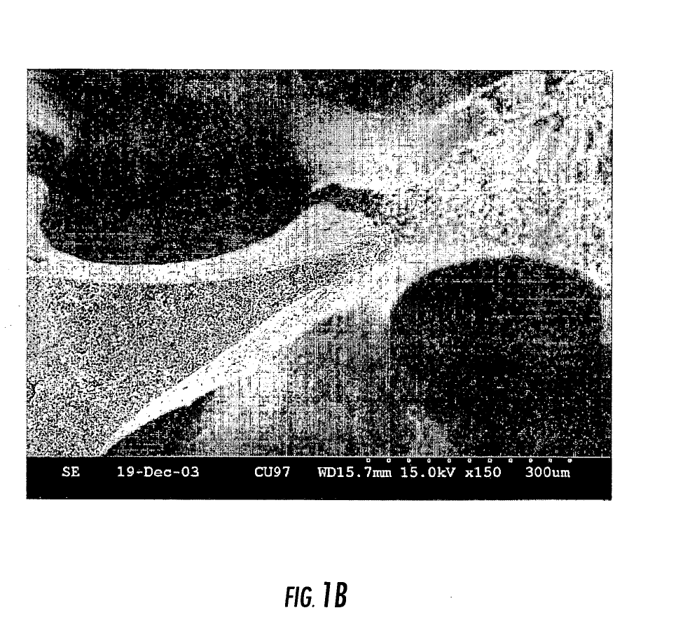

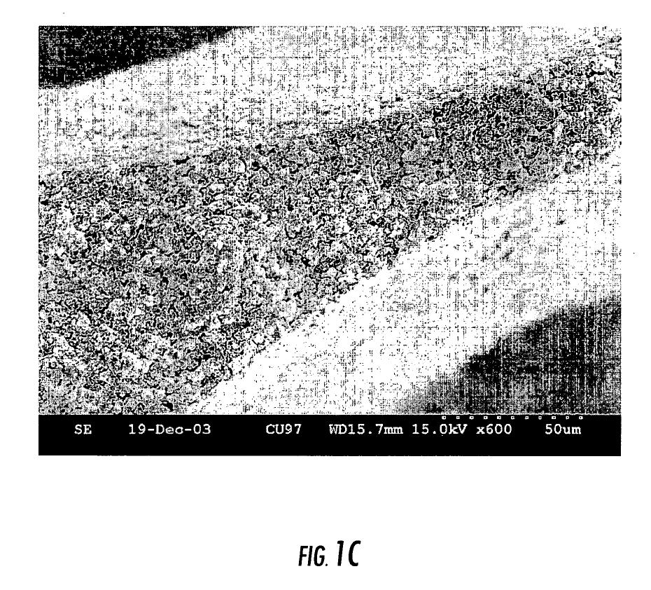

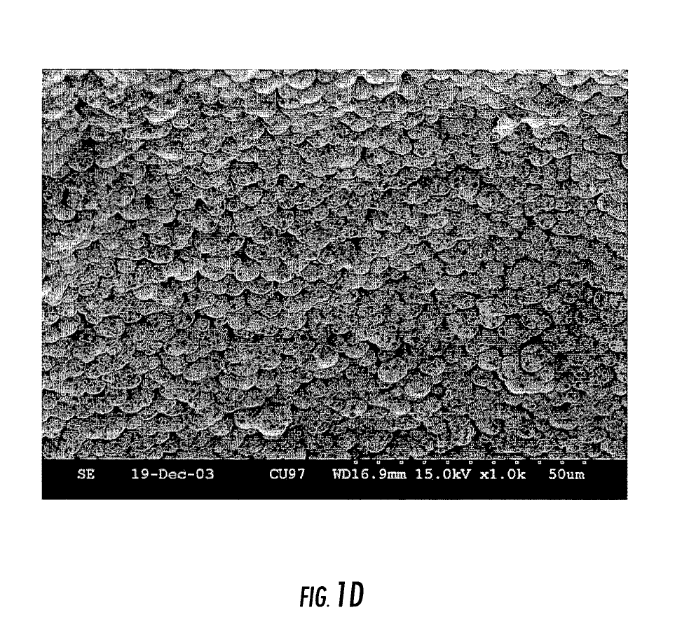

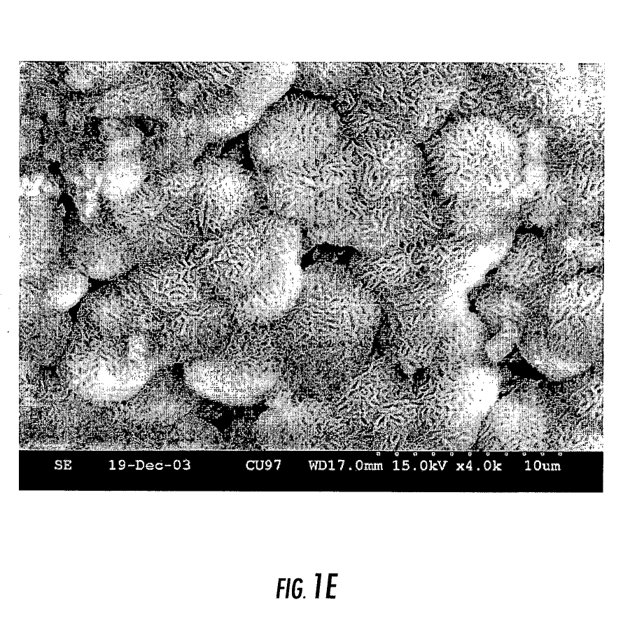

Among the reaction products that can form upon mixing the powdered and liquid components of the invention can be an amount of carbon dioxide. As the paste is mixed and cast into the final form of the scaffold or optionally applied to a target location as a bone cement, the released carbon dioxide can form an interconnected porous network throughout the paste. In particular, the interconnected porous network formed in the paste due to the release of carbon dioxide can include both microporosity and macroporosity. For example, referring to FIGS. 1A-1E, electron microscope micrographs of increasing magnification of a scaffold formed according to the disclosed processes are illustrated. FIG. 1A, covering a width of approximately 1 mm, clearly shows the macroporous network throughout the scaffold. In general, the macropores of the disclosed materials can have a pore size between about 300 μm and about 900 μm, though formation of smaller and/or larger macropores is also possible according to the process. FIG. 1E, which spans a distance of approximately 10 μm, clearly illustrates the microporosity formed during the disclosed process. The micropores of the disclosed materials can generally define a pore size between about 1 μm and about 10 μm, though again, as in the case of the macropores, larger and/or small micropore sizes are also possible.

Beneficially, the physical structure of the formed materials due to the interconnected porous network including both macro- and microporosity can closely resemble that of natural bone. The porous structure of the disclosed materials can exhibit high wicking ability, which can encourage infiltration of the materials by mesenchymal cells, fibroblasts and osteoblasts following implantation of the materials. This, when combined with the bone-like chemical composition of the materials, can provide excellent osseointegration capabilities to the materials.

In addition, the porous structure of the materials can enable the materials to be loaded with a biologically active material prior to implantation. For example, in one embodiment, the cured scaffold materials can be immersed in a solution including a biologically active material. Upon immersion, and in particular due to the wicking ability of the scaffold, the biologically active material can be loaded into the scaffold. The scaffold, now carrying the biologically active material can then be implanted in a patient, for example, in a bone defect caused due to trauma. The biological materials loaded into the scaffold can then be delivered to a patient over a period of time. Biologically active materials that can be delivered to a patient in such a manner can include, for example, treatment agents such as antibiotics, chemotherapeutic compounds, such as for cancer treatment, bone growth factors, and the like. In one embodiment, transplantable materials such as bone marrow and plasma rich platelets can be loaded into a scaffold and delivered to a patient according to the present invention.

The paste formed upon mixing the solid and liquid components according to the invention can include a phase mixture of partially crystalline, carbonated calcium hydroxyapatite and calcite as well as remaining sodium ions and any additives (e.g., ethanol or gelatin). In one embodiment, the paste can be applied to a target in this form and used as a bone cement. For example, the paste as formed can be used prior to cure to fill a bone defect caused by trauma or surgical procedure, as described above, or can be used to cement an implant, for example an artificial joint implant, in place during a joint replacement procedure. In another embodiment, the paste can be cast into a mold and allowed to cure to form a scaffold, for example a cube or prism-shaped scaffold, that can be shaped (e.g., cut) and/or implanted following cure of the paste. In another embodiment, a pre-formed mold of a particular shape can be utilized to exactly form the scaffold to desired specifications for a particular patient. For example, in one embodiment, the disclosed materials can be molded so as to fit in a particular location, such as, for instance, a prosthetic bone section for a particular patient. According to this embodiment, a mold with the exact dimensions can be prepared prior to the formation of the paste and the formed paste can then be cast into the mold for curing to the desired specifications.

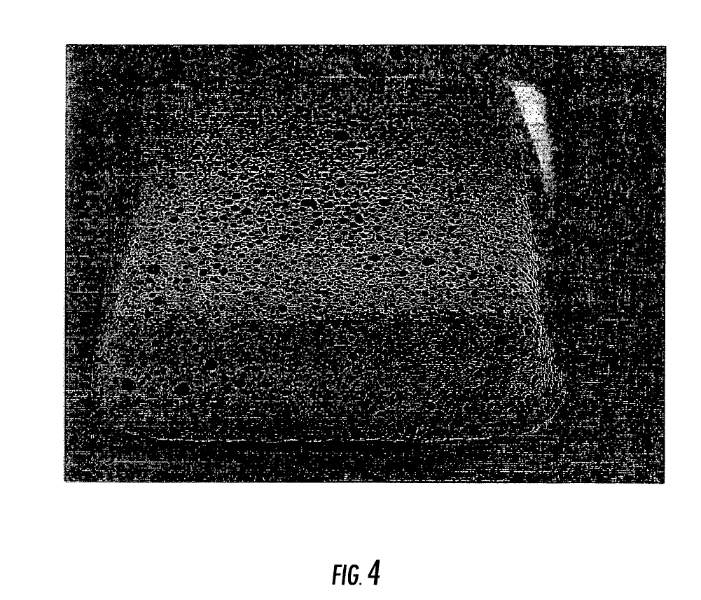

Following application or casting of the paste, the material can self-cure fairly rapidly, generally within about five minutes, at room temperature. FIG. 4 illustrates a fully cured and generally cubic-shaped scaffold of approximately 8 cm in width formed according to the disclosed process.

If desired, following complete cure, scaffolds formed according to the inventive process can be further processed. For example, in one embodiment, a cured scaffold can be soaked in water for a period of time. Soaking the scaffold in water can remove extraneous materials from the scaffold structure. For instance, any remaining carbon dioxide and sodium ions can be removed from the structure during this soaking step. In addition, in those embodiments wherein an additive has been added to the liquid component, for instance to delay cure of the paste, a soaking step can remove the additive from the scaffold. For example, a scaffold can be soaked in water for at least about one hour to remove additives. For example, in one embodiment, a scaffold can be soaked in water for a period of between about 10 hours and about 16 hours. In general, there is no need to heat or chill the water used in this soaking step, and the water can be at ambient temperature during the soaking process, though this is not a requirement of the invention, and in other embodiments, the water can be at a temperature other than ambient, if desired. According to one embodiment, following removal of any additives, the scaffold structure can include only carbonated hydroxyapatite and calcium carbonate. For example, the structure that forms the scaffold can be entirely a two-phase mixture of carbonated, partially crystallized calcium hydroxyapatite and calcium carbonate. Moreover, this two-phase mixture can be an essentially inseparable two-phase mixture as compared to, for instance, a two-phase mixture that includes separable phases, such as granules simply held in a matrix material.

In one embodiment, the cured scaffold can be further processed so as to more closely resemble the chemical make-up of bone mineral. According to this embodiment, the scaffold can be soaked, for instance in a sealed glass container, in a phosphate solution for a period of time so as to convert calcium carbonate remaining in the scaffold to hydroxyapatite. For example, according to one embodiment, following soaking the scaffold in a heated phosphate solution, the scaffold material can be entirely converted to hydroxyapatite, with no calcium carbonate remaining in the structure and the scaffold structure itself (which does not encompass any additional materials that can be carried by the scaffold in the porous network) can be formed entirely of carbonated, partially crystallized calcium hydroxyapatite.

The phosphate solution in which the scaffold can be soaked according to this embodiment can be an aqueous solution and can include sodium ions and phosphate ions. For instance, the soaking solution can be, in one embodiment, a solution of a sodium phosphate salt. The pH of this soaking solution can generally be near physiological pH (i.e., about pH 7.4). In one embodiment, this soaking solution can include the phosphate and sodium ions in chemically-balanced concentration so as to achieve the target pH. Optionally, this soaking solution can be buffered at the physiological pH by using a buffer as is generally known in the art. For instance, in one embodiment, the soaking solution can be buffered with PIPES (Piperazine-1,4-bis(2-ethanesulphonic acid)) buffer.

The soaking solution can optionally contain other materials as well. For example, the soaking solution can include an antibiotic to prevent bacterial growth within the soaking solution during the period of time the scaffold is in the soaking solution. For instance, the soaking solution can include small amounts (e.g., 0.01 to 0.04 g/L) NaN₃.

In one embodiment, the soaking solution can include a biologically active material for delivery to a patient via loading into the scaffold as previously described. For example, the soaking solution can include a treatment or preventative medication that can wick into the scaffold during the soaking period and can then be delivered to a patient following implantation of the scaffold.

The scaffold can be immersed in the soaking solution for a period of time during which the remaining calcium carbonate can be converted to more bone-like carbonated apatitic calcium phosphates. For example, a scaffold can be held in the soaking solution of a period of time of at least about 24 hours. In one embodiment, a scaffold can be held in the soaking solution for between about 24 hours and about 48 hours, for instance, for about 36 hours.

During this soaking step, the solution can also be heated. For example, the soaking solution can be heated to a temperature of between about 70° C. and about 100° C. In one embodiment, the soaking solution can be heated to a temperature of between about 70° C. and about 80° C., for instance to a temperature of about 80° C.

Following the soaking step of the process, the scaffolds of the present invention can be washed and dried. For example, the scaffolds can be washed in deionized water and dried at physiological temperature (about 37° C.).

Additional processing that can be carried out prior to implantation of the disclosed scaffolds can include sterilizing, packaging, shipping, and any final shaping of the scaffold. Additional processing steps such as these are generally known in the art, and thus are not described in detail herein.

Beneficially, as the disclosed processes can be carried out at low temperatures, and in particular at temperatures less than those at which carbonate ions could volatilize, the hydroxyapatite formed according to the invention can be carbonated hydroxyapatite and can more closely resemble the chemical nature of natural bone mineral as compared to many previously described hydroxyapatite scaffold materials. For example, in one embodiment, the disclosed materials can include hydroxyapatite having a carbonate ion concentration of between about 1% and about 10% by weight. In one embodiment, the disclosed materials can include a carbonate ion concentration of between about 2% and about 6% by weight. As such, the materials can closely mimic natural bone in chemical make-up and as such, can exhibit high in vivo resorbability as well as being capable of taking part in bone remodeling processes.

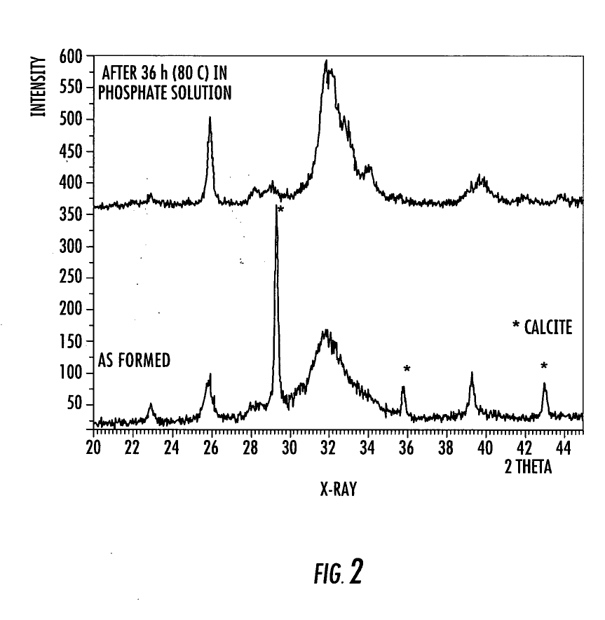

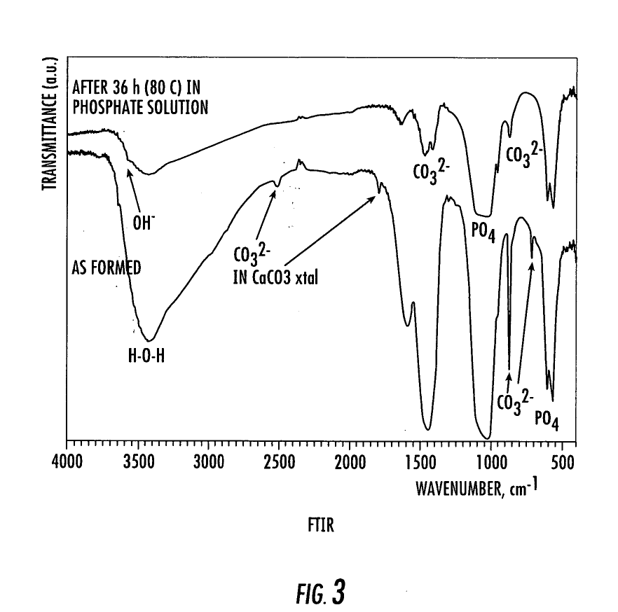

FIGS. 2 and 3 illustrate exemplary physical characteristics of materials formed according to the processes of the present invention. In particular, FIG. 2 illustrates X-ray diffraction data and FIG. 3 illustrates FTIR Spectroscopy data of carbonated calcium hydroxyapatite scaffolds formed according to the presently disclosed processes. The bottom trace in FIG. 2 displays the CaCO₃ (calcite) peaks still present in the as-formed porous scaffold. The XRD peaks observed for calcite conform well to the standardized powder diffraction file card No: 5-586 published by ICDD® (International Centre for Diffraction Data®, Newtown Square, Pa.). The remaining broad peaks are those of carbonated apatitic calcium phosphate (ICDD® PDF No: 9-432). These broad peaks very closely resemble those of natural bone mineral itself. The top trace is that of the same material following a period of soaking in a heated phosphate solution as described above. As can be seen, these date indicate the effectiveness of this soaking step in converting the CaCO₃ phase into carbonated apatite. The FTIR data shown in FIG. 3 exhibits the characteristic bands of carbonate ions originating from the phase CaCO₃ (in the bottom trace of “as-formed” scaffold), and then the removal of those during the soaking step (the top trace.) The top trace matches with the FTIR spectra of human bone mineral.

In one embodiment, the disclosed invention is directed to systems that can be utilized to carry out the disclosed methods. For example, in one embodiment, the invention is directed to self-contained systems that can include all of the materials necessary for carrying out the disclosed methods.

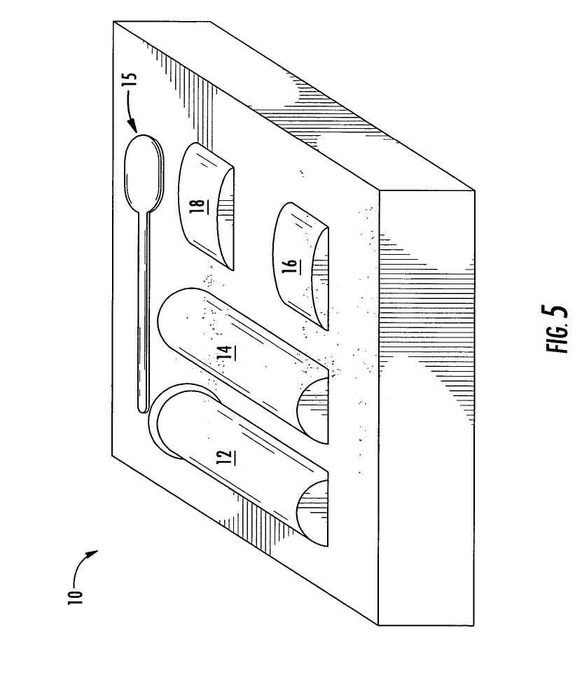

One embodiment of an exemplary system according to the present invention is illustrated in FIG. 5. As can be seen, the system generally 10 can include a container 12 for carrying an amount of a calcium carbonate powder such as, for example, calcite. The system 10 can also include a container 14 for carrying an amount of a liquid component including water, sodium ions, and phosphate ions in an amount suitable for mixing with the calcium carbonate powder carried in container 12 as herein described. Optionally, the system 10 can also include a mixing vial 16 in which all or a portion of the contents of container 12 and container 14 can be combined and mixed, as with mixing element 15. Mixing element 15 can be of any suitable construction for mixing the aqueous solution with the calcium carbonate powder. For example, mixing element can be a spatula, a spoon, a stick, a blade, or the like. Optionally, container 12 and/or container 14 can be designed with suitable volume so as to serve as a mixing vial during the formation process. Upon mixing the contents of container 12 with the contents of container 14 the paste as previously described herein can form.

In one embodiment, following mixing, the paste can be applied to a target tissue or implantable device prior to cure. In another embodiment, the paste can be allowed to cure, for example in the mixing vial 16 or in an optionally provided mold (not shown) and the system can be used to form a cured scaffold conforming to the shape of the mold. For instance, in one embodiment, a mold including a Teflon™ or nalgene-based polymeric surface can be provided, though there are no specific requirements for the surface properties of any suitable mold. Optionally, the system 10 can also include a container 18 for carrying a phosphate solution at about physiological pH as herein described for soaking the cured scaffold to convert remaining calcium carbonate in the scaffold to hydroxyapatite.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged, both in whole or in part. Therefore, the spirit and scope of the appended claims should not be limited to the description of the preferred versions contained therein.

What is claimed is:

1. A method for forming carbonated hydroxyapatite comprising:
providing a calcium carbonate powder;
providing a stable aqueous solution comprising phosphate ions, wherein the aqueous solution has a pH between about 3 and about 6;
combining the calcium carbonate powder with the aqueous solution at a ratio of between about 20 grams calcium carbonate powder per 30 milliliters aqueous solution and about 35 grams calcium carbonate powder per 30 milliliters aqueous solution; and
mixing the combination of the calcium carbonate powder and the aqueous solution for a period of time of at least about 30 seconds, wherein upon combination and mixing of the calcium carbonate powder with the aqueous solution, carbon dioxide and a paste are formed, the paste comprising a phase mixture of carbonated hydroxyapatite and calcium carbonate.

2. The method according to claim 1, further comprising applying the paste to a surface as a bone cement.

3. The method according to claim 2, wherein the bone cement cures within about five minutes of application.

4. The method according to claim 1, wherein the calcium carbonate powder is calcite.

5. The method according to claim 1, wherein the calcium carbonate powder is combined with the aqueous solution at a ratio of about 1 gram calcium carbonate powder per 1 milliliter aqueous solution.

6. The method according to claim 1, wherein the combination of the calcium carbonate powder and the aqueous solution are mixed for a period of time between about 30 seconds and about 90 seconds.

7. The method according to claim 1, wherein the aqueous solution further comprises an additive.

8. The method according to claim 7, wherein the additive is selected from the group consisting of ethanol and denatured collagen.

9. The method according to claim 7, wherein the combination of the calcium carbonate powder and the aqueous solution are mixed for a period of time between about 30 seconds and about 240 seconds.

10. The method according the claim 1, further comprising forming an interconnected porous network throughout the paste, the interconnected porous network defining both microporosity and macroporosity, wherein the interconnected network is formed by the carbon dioxide.

11. The method according to claim 1, further comprising casting the paste into a mold.

12. The method according to claim 11, further comprising curing the paste to form a scaffold, wherein the paste self-cures at ambient temperature within about five minutes of casting.

13. The method according to claim 12, further comprising soaking the scaffold in water for at least about one hour.

14. The method according to claim 13, wherein the scaffold is soaked in water at ambient temperature for between about 10 hours and about 16 hours.

15. The method according to claim 12 further comprising:
providing a second aqueous solution comprising phosphate ions, wherein the second aqueous solution has about a physiological pH;
heating the second solution to a temperature of between about 70° C. and about 100° C.; and
soaking the scaffold in the heated second solution.

16. The method according to claim 15, wherein the scaffold is soaked in the second solution for at least about 24 hours.

17. The method according to claim 15, wherein the scaffold is soaked in the second solution for between about 24 hours and about 48 hours.

18. The method according to claim 15, wherein the second aqueous solution further comprises a buffer.

19. The method according to claim 15, wherein the second aqueous solution further comprises a biologically active agent.

20. The method according to claim 19, the method further comprising loading the biologically active agent into the scaffold as the scaffold soaks in the heated second solution.

21. The method according to claim 12, further comprising packaging the scaffold for storage, shipping, or both.

22. The method according to claim 12, further comprising cutting the scaffold for implantation in a bone defect.

23. The method according to claim 12, further comprising implanting the scaffold in a bone defect.

24. A scaffold comprising a macroporous scaffold structure comprising carbonated, partially crystalline hydroxyapatite, the scaffold structure defining an interconnected porous network comprising both microporosity and macroporosity, wherein the scaffold structure defines a minimal cross sectional dimension of at least about one centimeter.

25. The scaffold of claim 24, wherein the microporosity comprises pores of between about 1 μm and about 10 μm in size.

26. The scaffold of claim 24, wherein the macroporosity comprises pores of between about 300 μm and about 900 μm in size.

27. The scaffold of claim 24, further comprising a biologically active agent loaded into the porous network of the scaffold structure.

28. The scaffold of claim 24, wherein the carbonated, partially crystalline hydroxyapatite comprises between about 1% and about 10% by weight carbonate ion.

29. The scaffold of claim 24, wherein the carbonated, partially crystalline hydroxyapatite comprises between about 2% and about 6% by weight carbonate ion.

30. The scaffold of claim 24, wherein the scaffold structure consists of a two phase mixture of the carbonated, partially crystalline hydroxyapatite and a second calcium carbonate phase.

31. The scaffold of claim 30, wherein the calcium carbonate is calcite.

32. The scaffold of claim 24, wherein the scaffold structure consists of a single phase of carbonated, partially crystalline hydroxyapatite.

33. The scaffold of claim 24, wherein the scaffold has been cured in vivo as a bone cement.

34. The scaffold of claim 24, wherein the scaffold has been cast and cured in a mold.

35. A system comprising:
a first container defining a first volume for holding a predetermined amount of a calcium carbonate powder;
a second container defining a second volume for holding a predetermined amount of an aqueous solution comprising phosphate ions, wherein the aqueous solution has a pH between about 3 and about 6; and
wherein the first volume and the second volume are such that the predetermined amounts of the calcium carbonate and the aqueous solution are provided in a ratio of between about 20 grams calcium carbonate powder per 30 milliliters aqueous solution and about 35 grams calcium carbonate powder per 30 milliliters aqueous solution.

36. The system of claim 35, further comprising a mixing element.

37. The system of claim 35, further comprising a third container for mixing the contents of the first container and the second container.

38. The system of claim 35, further comprising a fourth container for holding a predetermined amount of an aqueous soaking solution comprising phosphate ions, wherein the aqueous soaking solution has about a physiological pH, wherein the fourth container defines a volume for containing the mixed contents of the first container and the second container.

39. The system of claim 35, further comprising a mold for casting the mixed contents of the first container and the second container.
